(19) United States
(12) Patent Application Publication
(10) Pub. No.: US 2020/0140354 A1
(43) Pub. Date: May 7, 2020

(54) PROCESS FOR THE CONVERSION OF METHANOL TO P-XYLENE

(71) Applicant: BASF SE, Ludwigshafen Am Rhein (DE)

(72) Inventors: Robert MCGUIRE, Florham Park, NJ (US); Christiane KURETSCHKA, Ludwigshafen am Rhein (DE); Sven TITLBACH, Ludwigshafen am Rhein (DE); Ekkehard SCHWAB, Neustadt (DE); Ulrich MÜLLER, Ludwigshafen am Rhein (DE)

(21) Appl. No.: 16/607,532

(22) PCT Filed: Apr. 24, 2018
(86) PCT No.: PCT/EP2018/060444

§ 371 Date: Oct. 23, 2019

(30) Foreign Application Priority Data
Apr. 24, 2017 (EP) 17167770.1

(51) Int. Cl. C07C 1/20; B01J 29/40; C07C 15/08

(52) U.S. Cl. CPC C07C 1/20; C07C 2529/40; C07C 15/08; B01J 29/405

(57) ABSTRACT

The present invention relates to a process for the conversion of methanol in p-xylene comprising the use a molding which comprises a zeolitic material, phosphorous, one or more metals M of the groups 3, 6, 10 to 14 of the periodic system of the elements, and a binder material.

The present invention relates to a process for the conversion of methanol in p-xylene with high yield. The process comprises the use of a catalyst comprising, preferably consisting of a molding wherein the molding comprises a zeolitic material, phosphorous, one or more metals M of the groups 3, 6, 10 to 14 of the periodic system of the elements, and a binder material.

P-xylene is an aromatic compound useful in the production of terephtalic acid (PTA) and hence in the production of polyethylene terephthalate (PET). The p-xylene market has seen a strong growth due to the increasing interest in PET and in the intermediate in the preparation thereof such as PTA.

Conversion of methanol to aromatic compounds is known in the art. The conversion of methanol to aromatic compounds generally leads to a mixture of aromatic compounds known as BTX. BTX refers to mixtures of benzene, toluene and the three xylene isomers (p-xylene, m-xylene and o-xylene). In the BTX mixture p-xylene is comprised in a low amount. Due to the increasing interest in p-xylene is desirable to have a process that convert methanol in p-xylene in a high yield.

In view of this need, it was an object of the present invention to provide a process for the conversion of methanol in p-xylene wherein the reaction occurs in a high yield. The present inventors have surprisingly found that in the conversion reaction from methanol to BTX, p-xylene is obtained in high yield when using a molding comprising a zeolitic material and a binder material wherein the molding additionally comprises phosphorous and one or more metals M of the groups 3, 6, 10 to 14 of the periodic system of the element. The phosphorous and the one or more metals M of the groups 3, 6, 10 to 14 of the periodic system of the element impregnated the molding not only the zeolitic material.

Therefore, the present invention is directed to a process for preparing p-xylene, comprising

(I) providing a molding which comprises
(a) a zeolitic material,
(b) phosphorous,
(c) one or more metals M of the groups 3, 6, 10 to 14 of the periodic system of the elements, and
(d) a binder material;

(II) providing a gas stream comprising methanol;

(III) contacting the gas stream provided in (II) with the molding provided in (I), obtaining a reaction mixture comprising p-xylene.

### Step (I)

According to (I), a molding is provided. This molding serves, according to (III), as catalyst or as a catalyst component for the conversion of methanol, comprised in the gas stream provided in

(I), to p-xylene which is comprised in the reaction mixture obtained from (III). Said molding comprises

a zeolitic material, phosphorous, one or more metals M of the groups 3, 6, 10 to 14 of the periodic system of the elements, and a binder material. Surprisingly, it was found that the molding of the invention is particularly advantageous when used as a catalyst or as a catalyst component in a conversion reaction of methanol to p-xylene. In particular, it was found that if such a molding is prepared based on a molding comprising the zeolitic material by impregnating the latter molding with a source of the one or more metals M and a source of phosphorous, the conversion of methanol into aromatics occurs with a high yield.

Preferably, the molding comprises
(a) a zeolitic material,
(b) phosphorous,
(c) one or more metals M of groups 10 to 14 of the periodic system of the elements, and
(d) a binder material.

The framework structure of the zeolitic material according to a) preferably comprises YO₂ and X₂O₃, wherein Y is a tetravalent element and X is a trivalent element. Generally, no limitation exists as to the chemical nature of the tetravalent element Y. Preferably, Y is one or more of Si, Sn, Ti, Zr and Ge, more preferably Y is Si. Generally, no limitation exists to the chemical nature of the trivalent element X. Preferably, X is one or more of Al, B, In and Ga, more preferably X is Al. Therefore, it is preferred that Y is Si and X is Al.

Preferably, the zeolitic material has a molar ratio YO₂:X₂O₃ in the range of from 10 to 100, more preferably in the range of from 20 to 90, more preferably in the range of from 30 to 80, more preferably in the range of from 40 to 60, more preferably in the range of from 45 to 55.

Preferably at least 95 weight-%, more preferably at least 98 weight-%, more preferably at least 99 weight-%, more preferably at least 99.9 weight % of the framework structure of the zeolitic material consist of X, Y, O and H.

With regard to the zeolitic framework types, generally no specific restrictions exist. Generally, it is conceivable that the zeolitic framework type is one of ABW, ACO, AEI, AEL, AEN, AET, AFG, AFI, AFN, AFO, AFR, AFS, AFT, AFV, AFX, AFY, AHT, ANA, APC, APD, AST, ASV, ATN, ATO, ATS, ATT, ATV, AVL, AWO, AWW, BCT, BEA, BEC, BIK, BOF, BOG, BOZ, BPH, BRE,

BSV, CAN, CAS, CDO, CFI, CGF, CGS, CHA, -CHI, -CLO, CON, CSV, CZP, DAC, DDR, DFO, DFT, DOH, DON, EAB, EDI, EEI, EMT, EON, EPI, ERI, ESV, ETR, EUO, *-EWT, EZT, FAR, FAU, FER, FRA, GIS, GIU, GME, GON, GOO, HEU, IFO, IFR, -IFU, IFW, IFY, IHW, IMF, IRN, IRR, -IRY, ISV, ITE, ITG, ITH, *-ITN, ITR, ITT, -ITV, ITW, IWR, IWS, IWV, IWW, JBW, JNT, JOZ, JRY, JSN, JSR, JST, JSW, KFI, LAU, LEV, LIO, -LIT, LOS, LOV, LTA, LTF, LTJ, LTL, LTN, MAR, MAZ, MEI, MEL, MEP, MER, MFI, MFS, MON, MOR, MOZ, *MRE, MSE, MSO, MTF, MTN, MTT, MTW, MW, MWF, MWW, NAB, NAT, NES, NON, NPO, NPT, NSI, OBW, OFF, OKO, OSI, OSO, OWE, -PAR, PAU, PCR, PHI, PON, POS, PSI, PUN, RHO, -RON, RRO, RSN, RTE, RTH, RUT, RWR, RWY, SAF, SAO, SAS, SAT, SAV, SBE, SBN, SBS, SBT, SEW, SFE, SFF, SFG, SFH, SFN, SFO, SFS, *SFV, SFW, SGT, SIV, SOD, SOF, SOS, SSF, *-SSO, SSY, STF, STI, *STO, STT, STW, -SVR, SW, SZR, TER, THO, TOL, TON, TSC, TUN, UEI, UFI, UOS, UOV, UOZ, USI, UTL, UWY, VET, VFI, VNI, VSV, WEI, -WEN, YUG, ZON, or a mixed type of two or more thereof. More preferably, the zeolitic material comprises, more preferably is, one or more of zeolitic materials having a framework structure of type BEA, MFI, MWW, MEL, MOR, MTT, MTW, FER, TOL, and TON, more preferably the framework type is MFI, MWW, MEL, or TON.

Preferably, the zeolitic material comprises, more preferably is, one or more of a ZSM-5 zeolitic material, a ZSM-22 zeolitic material, a ZSM-11 zeolitic material, a ZBM-10 zeolitic material and a ZBM-11 zeolitic material. More preferably, the zeolitic material comprises, more preferably is, a ZBM-10 zeolitic material or a ZBM-22 zeolitic material.

The ZSM-22 zeolitic material, the ZSM-11 zeolitic material, the ZBM-10 zeolitic material and the ZBM-11 zeolitic material are known in the art. For example, the ZBM-10 zeolitic material is disclosed in patent application U.S. Pat. No. 4,401,636, the ZSM-22 zeolitic material is disclosed in “Journal of Catalysis, Vol. 147, Issue 2, June 1994, Pages 482-493” and the ZSM-5 zeolitic material is disclosed in patent application US 2014/0135556 A1.

According to b), the molding comprises phosphorous. With respect to the form in which the phosphorous is present in the molding, there is no particular restriction. Preferably at least a portion of the phosphorous is in oxidic form. Phosphorous is in oxidic form if at least a portion of the phosphorous is present as a chemical compound with oxygen, especially comprising a covalent bonding between the phosphorous and the oxygen. It is preferred that the phosphorous which is at least partly in oxidic form comprises oxides of phosphorous which include, but are not restricted to phosphorous trioxide, diphosphorous tetroxide, phosphorous pentoxide and a mixture of two or more thereof.

With regard to the amount of phosphorous in the molding according to the present invention, there is in general no restriction. It is preferred that the phosphorous is present in the molding in an amount of at least 0.1 weight-%, preferably in an amount in the range of from 0.1 to 5 weight-%, more preferably in the range of from 0.1 to 4 weight-%, more preferably in the range of from 0.1 to 3 weight-%, more preferably in the range of from 0.1 to 2 weight-%, calculated as elemental phosphorous and based on the total weight of the molding.

Hence, the molding preferably comprises
(a) a zeolitic material,
(b) phosphorous,
(c) one or more metals M of groups 3, 6 and 10 to 14 of the periodic system of the elements, preferably one or more metals M of groups 10 to 14 of the periodic system of the elements, and
(d) a binder material,

wherein the zeolitic material comprises, preferably is, one or more of a ZBM-10 zeolitic material and a ZSM-22 zeolitic material,

wherein the phosphorous is present in the molding in an amount in the range of from 0.1 to 5 weight-%, preferably in the range of from 0.1 to 2 weight-%, calculated as elemental phosphorous and based on the total weight of the molding.

According to c), the molding comprises one or more metals M of groups 3, 6, 10 to 14 of the periodic system of the elements, preferably one or more metals M of groups 10 to 14 of the periodic system of the elements. More preferably, the molding comprises one or more of Ni, Pd, Pt, Cu, Ag, Ar, Zn, Cd, Hg, B, Al, Ga, In, TI, C, Si, Ge, Sn and Pb, Mo and La. More preferably, the one or more metals M are one or more of Ga, Zn, Ni, Mo, La and Pt, more preferably one or more of Ga and Zn.

Hence, the molding preferably comprises
(a) a zeolitic material,
(b) phosphorous,
(c) one or more metals M of groups 3, 6, 10 to 14 of the periodic system of the elements, and
(d) a binder material

wherein the zeolitic material comprises, preferably is, one or more of a ZBM-10 zeolitic material and a ZSM-22 zeolitic material, preferably the zeolitic material is a ZBM-10 zeolitic material wherein the phosphorous is present in the molding in an amount in the range of from 0.1 to 5 weight-%, preferably in the range of from 0.1 to 2 weight-%, calculated as elemental phosphorous and based on the total weight of the molding,

wherein the one or more metals M is one or more of Ga and Zn.

Generally, there is no limitation as to the amount of the one or more metals M in the molding. Preferably, the molding comprises the one or more metals M, calculated as elemental M, in an amount of at least 1 weight-%, more preferably in an amount in the range of from 1 to 4 weight-%, more preferably in an amount in the range of from 1.25 to 3 weight-%, more preferably in the range of from 1.5 to 2.5 weight-%, based on the total weight of the molding, wherein said amount refers to the total amount of all metals M.

Hence, the molding preferably comprises
(a) a zeolitic material,
(b) phosphorous,
(c) one or more metals M of groups 3, 6 10 to 14 of the periodic system of the elements, and
(d) a binder material

wherein the zeolitic material comprises, preferably is, one or more of a ZBM-10 zeolitic material and a ZBM-11 zeolitic material, preferably the zeolitic material is a ZBM-10 zeolitic material, wherein the phosphorous is present in the molding in an amount in the range of from 0.1 to 5 weight-%, preferably in the range of from 0.1 to 2 weight-%, calculated as elemental phosphorous and based on the total weight of the molding,

wherein the molding comprises the one or more metals M, calculated as elemental M, in an amount in the range of from 1.5 to 2.5 weight-%, based on the total weight of the molding, wherein said amount refers to the total amount of all metals M,

wherein the one or more metals M is one or more of Ga and Zn.

It is preferred that, as discussed below, the molding is prepared by impregnation with the one or more of metals M. Therefore, it is preferred that with respect to the zeolitic material, the one or more metals M is comprised in the zeolitic material as extra-framework elements.

According to d), the molding further comprises a binder material. Possible binder materials include all materials which are known to those skilled in the art.

Preferably, the binder material is one or more of a graphite, a silica, a titania, a zirconia, an alumina, and a mixed oxide of two or more of silicon, titanium, aluminum and zirconium, preferably one or more of a graphite, a silica, a titania and a zirconia, wherein more preferably the binder material is a zirconia or a silica. The weight ratio of the zeolitic material in the molding relative to the binder material (weight(zeolitic material):weight(binder material)) is generally not subject to any specific restriction. Preferably, the weight ratio of the zeolitic material relative to the binder material is in the range of from 10:1 to 1:1, more preferably in the range of from 7:1 to 2:1. More preferably, it is in the range of from 5:1 to 3:1, more preferably in the range of from 4.5:1 to 3.5:1, more preferably in the range of from 4.1:1 to 3.9:1. More preferably, the weight ratio is 4:1.

Preferably at least 95 weight-%, more preferably at least 98 weight-%, more preferably at least 99 weight-%, more preferably at least 99.9 weight-% of molding consist of the zeolitic material, the phosphorous, oxygen, the one or more metals M and the binder material.

Hence, the molding preferably comprises
(a) a zeolitic material,
(b) phosphorous,
(c) one or more metals M of groups 3, 6 10 to 14 of the periodic system of the elements, and
(d) a binder material

wherein the zeolitic material comprises, preferably is, one or more of a ZBM-10 zeolitic material and a ZBM-11 zeolitic material, preferably the zeolitic material is a ZBM-10 zeolitic material wherein the phosphorous is present in the molding in an amount in the range of from 0.1 to 5 weight-%, preferably in the range of from 0.1 to 2 weight-%, calculated as elemental phosphorous and based on the total weight of the molding,

wherein the molding comprises the one or more metals M, calculated as elemental M, in an amount in the range of from 1.5 to 2.5 weight-%, based on the total weight of the molding,

wherein said amount refers to the total amount of all metals M and wherein the one or more metals M is one or more of Ga and Zn,

wherein the binder material is zirconia or silica, wherein the weight ratio of the zeolitic material relative to the binder material is preferably in the range of from 4.5:1 to 3.5:1.

The molding may be in any form suitable for its intended use. The molding may be a shaped body. The molding of the invention preferably has a rectangular, a triangular, a hexagonal, a square, an oval or a circular cross section, and/or preferably is in the form of a star, a tablet, a sphere, a cylinder, a strand, or a hollow cylinder.

Preferably, the molding has a total pore area in the range of 20 to 80 m²/g, more preferably in the range of from 25 to 50 m²/g, more preferably in the range of 30 to 40 m²/g determined as described in Reference Example 1 herein. More preferably, the molding of the invention has a total pore area in the range of from 33 to 37 m²/g, such as 35 m²/g.

Preferably, the molding has a BET (Brunauer-Emmett-Teller) specific surface area in the range of from 100 to 500 m²/g, more preferably in the range of from 100 to 450 m²/g, more preferably in the range of from 100 to 400 m²/g, more preferably in the range of from 100 to 350 m²/g, determined according to Reference Example 2 herein.

Preferably, the molding has a total intrusion volume in the range of from 0.15 to 3 mL/g, more preferably in the range of from 0.2 to 2.5 mL/g, more preferably in the range of from 0.3 to 2 mL/g, more preferably in the range of from 0.4 to 1 mL/g, more preferably in the range of from 0.5 to 0.85 mL/g, determined as described in Reference Example 3 herein.

According to the present invention, the molding is preferably a calcined molding. Preferably the molding is a molding have been calcined under a gas atmosphere having a temperature in the range of from 400 to 750° C., more preferably in the range of from 450 to 650° C., more preferably in the range of from 500 to 550° C., wherein said gas atmosphere preferably comprises oxygen, said gas atmosphere more preferably being air.

Preferably, the molding comprises micropores, having a pore size in the range of less than 2 nm, and further comprises mesopores, having a pore size in the range of from 2 to 50 nm.

According to the invention, it is further preferred that providing the molding according to (I) comprises or consists of preparing the molding.

Preferably, preparing the molding comprises
(i) providing the zeolitic material,
(ii) mixing the zeolitic material provided in (i) with a source of the binder material,
(iii) subjecting the mixture obtained from (ii) to molding,
(iv) impregnating the molding obtained from (iii) with a source of the one or more metals M of groups 3, 6, 10 to 14 of the periodic system of the elements and a source of the phosphorous.

The zeolitic material provided according to (i) is as defined hereinabove. Therefore, it is preferred that preparing the molding comprises
(i) providing the zeolitic material
(ii) mixing the zeolitic material provided in (i) with a source of the binder material
(iii) subjecting the mixture obtained from (ii) to molding
(iv) impregnating the molding obtained from (iii) with a source of the one or more metals M of groups 3, 6, 10 to 14 of the periodic system of the elements and a source of the phosphorous

wherein the zeolitic material comprises, preferably is, one or more of a ZBM-10 zeolitic material and a ZBM-11 zeolitic material, preferably the zeolitic material is a ZBM-10 zeolitic material.

According to (ii), the zeolitic material provided in (i) is mixed with a source of the binder material. The binder material is as defined above in the corresponding paragraph. Possible sources of the binder materials include all materials which are known to those skilled in the art and can be used here as said sources. Preferably, the source of the binder material is chosen so that in the finally obtained molding, the binder is one or more of a graphite, a silica, a titania, a zirconia, an alumina, and a mixed oxide of two or more of silicon, titanium and zirconium, preferably one or more of a graphite, a silica, a titania and a zirconia, more preferably the binder material is a zirconia or a silica. The weight ratio of the zeolitic material relative to the source of the binder material mixed according to (ii) is generally not subject to any specific restrictions. Preferably, the weight ratio is chosen so that in the finally obtained molding, the weight ratio of the zeolitic material relative to the binder material is in the range of from 10:1 to 1:1, more preferably in the range of from 7:1 to 2:1. More preferably, it is in the range of from 5:1 to 3:1, more preferably in the range of from 4.5:1 to 3.5:1, more preferably in the range of from 4.1:1 to 3.9:1. More preferably, the weight ratio is 4:1. If the binder material is silica, the source of the binder material preferably comprises one or more of a colloidal silica, a silica gel and a waterglass, more preferably a colloidal silica.

Therefore, it is preferred that preparing the molding comprises
(i) providing the zeolitic material
(ii) mixing the zeolitic material provided in (i) with a source of the binder material
(iii) subjecting the mixture obtained from (ii) to molding
(iv) impregnating the molding obtained from (iii) with a source of the one or more metals M of groups 3, 6, 10 to 14 of the periodic system of the elements and a source of the phosphorous

wherein the zeolitic material comprises, preferably is, one or more of a ZBM-10 zeolitic material and a ZBM-11 zeolitic material, preferably the zeolitic material is a ZBM-10 zeolitic material, wherein the source of the binder material comprises one or more of a colloidal silica, a silica gel and a waterglass, preferably is a colloidal silica.

Generally it is conceivable that according to (ii), in addition to one or more zeolitic materials provided in (i) and the one or more source of the binder material, one or more additional agents may be provided in (ii). The additional agent can be one or more of a kneading agent and a pore forming agent. The pore forming agent is preferably a mesopore forming agent.

Hence, in (ii), a kneading agent may be further added to the mixture comprising the zeolitic material and the source of binding material. According to the invention, there is no limitation as to the kneading agent. The kneading agent is preferably a polar protic kneading agent, more preferably one or more of water, alcohols, and mixtures of two or more thereof, more preferably one or more of water, C1-C5 alcohols, and mixtures of two or more thereof, more preferably one or more of water, C1-C4 alcohols, and mixtures of two or more thereof, more preferably one or more of water, methanol, ethanol, propanol, and mixtures of two or more thereof, wherein more preferably, the kneading agent comprises, more preferably is water.

Further according to the present invention, there is no limitation as to the amount of kneading agent provided that the molding is obtained. Preferably, in the mixture obtained from (ii), the weight ratio of the kneading agent relative to the zeolitic material is in the range of from 0.5:1 to 2:1, more preferably in the range of from 0.75:1 to 1.7:1, more preferably in the range of from 1.0:1 to 1.5:1.

Further according to (ii), the zeolitic material provided in (i) is mixed with a source of the binder material and a mesopore forming agent and preferably the kneading agent.

A “mesopore forming agent” is a compound that assists the formation of pores having a diameter in the range of from 2 to 50 nm.

Generally, there is no specific limitation as to the chemical nature mesopore forming agent. Preferably the mesopore forming agent is one or more of polymers, carbohydrates, graphite, and mixtures of two or more thereof. More preferably, the mesopore forming agent is one or more of polymeric vinyl compounds, polyalkylene oxides, polyacrylates, polyolefins, polyamides, polyesters, cellulose, cellulose derivatives, sugars, and mixtures of two or more thereof, more preferably one or more of polystyrene, polyethylene oxides, polypropylene oxides, cellulose derivatives, sugars, and mixtures of two or more thereof, more preferably one or more of polystyrene, polyethylene oxide, C1-C2 hydroxyalkylated and/or C1-C2 alkylated cellulose derivatives, sugars, and mixtures of two or more thereof, more preferably one or more of polystyrene, polyethylene oxide, hydroxyethyl methyl cellulose, and mixtures of two or more thereof. More preferably, the mesopore forming agent comprises, more preferably is, one or more of polyethylene oxide and hydroxyethyl methyl cellulose.

Generally there is no particular limitation as to the amount of mesopore forming agent. Preferably, the weight ratio of the mesopore forming agent relative to the zeolitic material in the mixture according to (ii) is in the range of from 0.001:1 to 0.3:1, more preferably in the range of from 0.005:1 to 0.1:1, more preferably in the range of from 0.01:1 to 0.05:1, more preferably in the range of from 0.02:1 to 0.04:1, more preferably in the range of from 0.025:1 to 0.035:1.

According to the present invention, it is further preferred that the one or more of the kneading material and the mesopore forming agent are not part of the final molding. If the kneading material and/or the mesopore forming agent remain in the final molding, they may remain as impurity only. It is further contemplated that the one or more kneading agents and mesopore forming agents are preferably removed by calcination preferably after step (iii) as disclosed herein below.

Therefore, it is preferred that preparing the molding comprises
(i) providing the zeolitic material
(ii) mixing the zeolitic material provided in (i) with a source of the binder material
(iii) subjecting the mixture obtained from (ii) to molding
(iv) impregnating the molding obtained from (iii) with a source of the one or more metals M of groups 3, 6, 10 to 14 of the periodic system of the elements and a source of the phosphorous

wherein the zeolitic material comprises, preferably is, one or more of a ZBM-10 zeolitic material and a ZBM-11 zeolitic material, preferably the zeolitic material is a ZBM-10 zeolitic material,

wherein the source of the binder material comprises one or more of a colloidal silica, a silica gel and a waterglass, preferably is a colloidal silica

wherein in (ii) the zeolitic material is mixed with a kneading agent and/or a mesopore forming agent, wherein the zeolitic material is preferably mixed with a kneading agent and a mesopore forming agent.

According to (iii), the process further comprises subjecting the mixture obtained from (ii) to molding.

Generally, as far as (iii) is concerned, no specific restrictions exist. Preferably, the subjecting of the mixture from (ii) to molding according to (iii) comprises shaping the mixture of (ii) and obtaining a molding.

Depending on the chosen geometry of the molding which usually is adapted to the intended use of the molding, the shaping process according to (iii) will be chosen. If strands are prepared, the shaping according to (iii) preferably comprises subjecting the mixture obtained in (ii) to extrusion. Suitable extrusion apparatuses are described, for example, in “Ullmann's Enzyklopadie der Technischen Chemie”, 4th edition, vol. 2, page 295 et seq., 1972. If necessary, the extruder can be suitably cooled during the extrusion process. The strands leaving the extruder via the extruder die head can be mechanically cut by a suitable wire or via a discontinuous gas stream.

The molding obtained from shaping such as from extrusion is preferably dried and/or calcined after (iii) and prior to (iv). No specific restrictions exist concerning drying and calcination conditions. Preferably after the drying, the molding obtained is subjected to calcining.

The drying is preferably carried out in a gas atmosphere having a temperature in the range of from 50 to 200° C., more preferably in the range of from 75 to 150° C., more preferably in the range of from 100 to 125° C. The drying step is carried out for the time necessary to obtain a dried molding. Preferably, the duration of the drying is in the range of from 6 to 24 h, more preferably in the range of from 10 to 20 h. The drying can be effected under any suitable gas atmosphere such as air, lean air, or nitrogen such as technical nitrogen, wherein air and/or lean air are preferred.

Generally, as far as the calcination is concerned, no specific restrictions exist. Preferably, the calcination of is carried out under a gas atmosphere having a temperature in the range of from 400 to 750° C., more preferably in the range of from 450 to 650° C., more preferably in the range of from 500 to 550° C. The gas atmosphere preferably comprises oxygen, said gas atmosphere preferably is air. Preferably the calcination has a duration in the range of from 0.25 to 6 h, more preferably in the range of from 0.5 to 2 h.

According to (iv), the process of the invention comprises impregnating the molding obtained from (iii) with a source of the one or more metals M of groups 3, 6 10 to 14 of the periodic system of the elements and a source of the phosphorous.

Preferably, the impregnating according to (iv) comprises impregnating the molding with the source of the one or more metals M and the source of the phosphorous. The impregnating with the source of the one or more metals M of groups 3, 6 10 to 14 of the periodic system of the elements and a source of the phosphorous is carried out in sequence or simultaneously. It is preferred that the impregnating with the source of the one or more metals M and the source of the phosphorous is carried out in sequence. It is preferred that impregnating with the source of the one or more metals M is carried out prior to the impregnating with the source of the phosphorous.

The source of one or more metals M of groups 3, 6 10 to 14 of the periodic system of the elements and/or the source of phosphorous are applied in the form of aqueous, organic or organicaqueous solutions of the source by impregnating the molding with a respective solution. The impregnation can be carried out by spray impregnation by spraying the molding with a solution comprising the source of one or more metals M and/or the source of phosphorous. The impregnation can also be carried out by the incipient wetness method in which the porous volume of the molding is filled with a certain, in some cases an approximately equal volume of impregnation solution. It is also possible to employ an excess of solution, in which case the volume of this solution is greater than the porous volume of the molding. In this case, the molding is mixed with the impregnation solution and stirred for a sufficiently long time. Other impregnation methods known to those skilled in the art are also possible. It is preferred that the impregnation is carried out by spraying the molding comprising the source of one or more metals M and/or the source of phosphorous.

The solution comprising the source of phosphorous is preferably an aqueous, organic, or organic-aqueous solution. More preferably, the solution comprising the source of phosphorous is an aqueous solution. More preferably, the water of the aqueous solution is deionized water.

The solution comprising the source of the one or more metals M is preferably an aqueous, organic, or organic-aqueous solution. More preferably, the solution comprising the source of the one or more metals M is an aqueous solution. More preferably, the water of the aqueous solution is deionized water.

Hence, (iv) preferably further comprises preparing a solution comprising the source of the one or more metals M and/or the source of the phosphorous and impregnating the molding obtained from (iii) with said solution or solutions, wherein preferably the impregnating comprises, more preferably consists of is a spray impregnation.

Hence, (iv) preferably further comprises preparing a solution comprising the source of the one or more metals M and/or the source of phosphorous, or a solution comprising the source of the one or more metals M and a solution comprising the source of the phosphorous. Preparing the solution or the solutions preferably comprises suitably dissolving the source of the one or more metals M and/or the source of the phosphorous in water, preferably deionized water.

Therefore, (iv) preferably comprises
(iv-1) impregnating the molding with the source of the one or more metals M of groups 3, 6 10 to 14 of the periodic system of the elements;
(iv-2) impregnating the molding obtained from (iv-1) with the source of phosphorous,

wherein the impregnating of (iv-1) and/or (iv-2) preferably comprises spray impregnating.

After the impregnation of (iv-1) as disclosed above the molding is preferably dried. The drying is preferably carried out in a gas atmosphere having a temperature in the range of from 50 to 200° C., more preferably in the range of from 75 to 150° C., more preferably in the range of from 100 to 125° C. more preferably in the range from about 80 to 130° C., usually for a duration in the range of from 4 to 20 hours under reduced pressure. The gas atmosphere preferably comprises oxygen, preferably is air.

Preferably after (iv-1), preferably after the drying, the molding obtained is subjected to calcining. Generally there is no restriction as to the calcining conditions. The calcining is preferably carried out in a gas atmosphere having a temperature in the range of from 400 to 750° C., more preferably in the range of from 450 to 650° C., more preferably in the range of from 500 to 550° C. The gas atmosphere preferably comprises oxygen, preferably is air. Preferably the calcination has a duration in the range of from 0.25 to 6 h, more preferably from 0.5 to 2 h.

After (iv-1), preferably after the drying, preferably after the calcining the molding obtained is further impregnated with a source of phosphorous.

After the impregnation of (iv-2) as disclosed above the molding is preferably dried. The drying is carried out in a gas atmosphere having a temperature in the range of from 50 to 200° C., preferably in the range of from 75 to 150° C., more preferably in the range of from 100 to 125° C. more preferably in the range from about 80 to 130° C., usually for a duration in the range of from 4 to 20 hours under reduced pressure. The gas atmosphere preferably comprises oxygen, preferably is air.

Preferably after (iv-2), preferably after the drying, the molding obtained is subjected to calcining. Generally there is no restriction as to the calcining conditions. The calcining preferably is carried out in a gas atmosphere having a temperature in the range of from 400 to 750° C., preferably in the range of from 450 to 650° C., more preferably in the range of from 500 to 550° C. The gas atmosphere preferably comprises oxygen, preferably is air. Preferably the calcination has a duration in the range of from 0.25 to 6 h, more preferably from 0.5 to 2 h.

Therefore, it is preferred that preparing the molding comprises
(i) providing the zeolitic material
(ii) mixing the zeolitic material provided in (i) with a source of the binder material
(iii) subjecting the mixture obtained from (ii) to molding
(iv) impregnating the molding obtained from (iii) with a source of the one or more metals M of groups 3, 6, 10 to 14 of the periodic system of the elements and a source of the phosphorous

wherein the zeolitic material comprises, preferably is, one or more of a ZBM-10 zeolitic material and a ZBM-11 zeolitic material, preferably the zeolitic material is a ZBM-10 zeolitic material wherein (iv) comprises
(iv-1) impregnating the molding with the source of the one or more metals M of groups 3, 6 10 to 14 of the periodic system of the elements;
(iv-2) impregnating, the molding obtained from (iv-1) with the source of phosphorous, wherein preferably the impregnating of (iv-1) and/or (iv-2) is a spray impregnating.

Steps (iv-1) and (iv-2) as disclosed above can be carried out in a reverse sequence. Hence, step (iv) can comprises
(iv-1′)impregnating the molding with the source of the phosphorous
(iv-2′)impregnating the molding obtained from (iv-1′) with the source of the one or more metals M of groups 3, 6 10 to 14 of the periodic system of the elements.

The impregnating of (iv-1′) is carried out as disclosed above for the impregnating of (iv-2). It is preferred that the drying is carried out by spray impregnation. The drying and the calcining after step (iv-1′) are carried out as the drying and the calcining after step (iv-2) as disclosed above.

The impregnating of (iv-2′) is carried out as disclosed above for the impregnating of (iv-1). It is preferred that the drying is carried out by spray impregnation. The drying and the calcining after step (iv-2′) are carried out as the drying and the calcining after step (iv-1) as disclosed above.

As to the source of the one or more metals M, there is no particular restriction. Preferably the source of the one or more metals M of groups 3, 6 10 to 14 of the periodic system of the elements is a salt or a complex of said one or more metals M. As to the salts it is preferred that the salts are one or more of inorganic salts or organic salts, more preferably one or more inorganic salts of said one or more metals M of groups 3, 6 10 to 14 of the periodic system of the elements, more preferably a bromide, a chlorate, a chloride, an iodide, a nitrate, or a sulfate of said one or more metals M of groups 3, 6 10 to 14 of the periodic system of the elements. It is more preferred that the source of the one or more metals M of groups 3, 6 10 to 14 of the periodic system of the elements is a nitrate.

According to the present invention, preferably the one or more metals M of groups 3, 6 10 to 14 of the periodic system of the elements comprises, more preferably is, Zn and the source of Zn is an inorganic salts of zinc(II), wherein preferably the inorganic salts of zinc(II) is zinc(II), nitrate.

According to the present invention, preferably the one or more metals M of groups 3, 6 10 to 14 of the periodic system of the elements comprises, more preferably is Ga and the source of Ga is an inorganic salts of gallium(III), wherein preferably the inorganic salts of gallium (iii) is gallium (IIII) nitrate.

Therefore, it is preferred that preparing the molding comprises
(i) providing the zeolitic material;
(ii) mixing the zeolitic material provided in (i) with a source of the binder material;
(iii) subjecting the mixture obtained from (ii) to molding;
(iv) impregnating the molding obtained from (iii) with a source of the one or more metals M of groups 3, 6, 10 to 14 of the periodic system of the elements and a source of the phosphorous

wherein the zeolitic material comprises, preferably is, one or more of a ZBM-10 zeolitic material and a ZBM-11 zeolitic material, preferably the zeolitic material is a ZBM-10 zeolitic material wherein (iv) comprises
(iv-1) impregnating the molding with the source of the one or more metals M of groups 3, 6 10 to 14 of the periodic system of the elements;
(iv-2) impregnating, the molding obtained from (iv-1) with the source of phosphorous,

wherein preferably the impregnating of (iv-1) and/or (iv-2) is a spray impregnating

wherein the source of the one or more metals M of groups 3, 6, 10 to 14 of the periodic system of the elements is a salts of said one or more metal M, wherein preferably the salt is a bromide, a chlorate, a chloride, an iodide, a nitrate, or a sulfate of said one or more metals M, more preferably the salt is a nitrate salt and

wherein the one or more metals M of groups 3, 6, 10 to 14 of the periodic system of the elements is one or more of Ga and Zn.

As to the source of the source of the phosphorous there is no particular restriction. Preferably is one or more of phosphorous acid (H₃PO₃), phosphoric acid (H₃PO₄), a salt of phosphorous acid, a salt of phosphoric acid, and a dihydrogen phosphate anion containing compound, wherein the dihydrogen phosphate anion containing compound is preferably one or more of monoammonium phosphate and diammonium phosphate, wherein the source of the phosphorous is more preferably one or more of phosphorous acid (H₃PO₃) and phosphoric acid (H₃PO₄), more preferably is phosphoric acid.

### Step (II)

In step (II) a gas stream comprising methanol is provided.

Generally there is no particular limitation as to the amount of methanol in the gas stream, provided that p-xylene is formed with high yield. Preferably, the gas stream provided in (II) comprises methanol in an amount in the range of from 30 to 70 volume-%, more preferably in the range of from 35 to 65 volume-%, more preferably in the range of from 40 to 60 volume-%, more preferably in the range of from 45 to 57.5 volume-%, more preferably in the range of from 50 to 55 volume-%, based on the total volume of the gas stream.

Preferably the gas stream further comprises one or more inert gases. Preferably, at least 95 volume-% of the gas stream provided in (II) consist of methanol and the one or more inert gases. More preferably at least 98 volume-%, more preferably at least 99 volume-% of the gas stream provided in (II) consist of methanol and the one or more inert gases.

There is no specific limitation as to the one or more inert gas. It is preferred that the one or more inert gases are one or more of helium, neon, argon, nitrogen, carbon monoxide, and carbon dioxide, more preferably one or more of argon, nitrogen, and carbon dioxide, wherein more preferably, the one or more inert gases comprise nitrogen, wherein more preferably, the one or more inert gases is nitrogen or a mixture of nitrogen and argon. There is no specific limitation as to the volume ratio of the nitrogen relative to the argon. Preferably the volume ratio is in the range of from 2.5:1 to 5:1, more preferably in the range of from 3.5:1 to 4.5:1.

### Step (III)

According to (III), the molding provided in (I) and the gas stream provided in (II) are brought into contact.

Preferably the contacting of (III) is carried out at a temperature of the gas stream in the range of from 250 to 750° C., more preferably in the range of from 275 to 725° C., more preferably in the range of from 300 to 700° C., more preferably in the range of from 325 to 675° C., more preferably in the range of from 350 to 650° C. Preferred ranges include of from 350 to 450° C. or from 400 to 500° C. or from 450 to 550° C. of from 500 to 600° C. or from 550 to 650° C.

Preferably, the contacting according to (III) is effected at a pressure of the gas stream in the range of from 1 to 100 bar(abs), more preferably in the range of from 1.2 to 50 bar(abs), more preferably in the range of from 1.5 to 35 bar(abs). Preferred ranges include of from 1.5 to 10 bar(abs) or from 5 to 15 bar(abs) or from 10 to 20 bar(abs) or from 15 to 25 bar(abs) or from 20 to 30 bar(abs) or from 25 to 35 bar(abs).

It is further preferred that the contacting according to (III) is carried out in semi-continuous mode or in continuous mode, preferably in continuous mode.

As to the space velocity (gas hourly space velocity, GHSV) with respect to the contacting in (III), it is preferably chosen such that an advantageous balance of conversion, selectivity, yield, reactor geometry, reactor dimensions and process regime is obtained. In the context of the present invention, the gas hourly space velocity is defined as the volume flow of methanol comprised in the gas stream provided in (II) and subjected to (III) in L/h divided by the volume of the catalyst (here: molding) present in the catalyst bed of the reactor in L with which the mixture provided in (II) contacted in (III). The gas hourly space velocity therefore has the unit h⁻¹. Preferably, the GHSV in the present process is in the range of from 500 to 3,000 h⁻¹, more preferably in the range of from 1,000 to 2,500 h⁻¹, more preferably in the range of from 1,000 to 1,600 h⁻¹.

From (III), a reaction mixture comprising p-xylene is obtained. Advantageously, according to the process of the invention, p-xylene is obtained in a yield of at least 5.5%, preferably in a yield in the range of from 5.5 to 40%, more preferably in the range of from 8 to 35%. The yield referred to is to be understood as being defined in Reference Example 4 herein.

If so intended, the reaction mixture obtained from (III) can be suitably purified, in particular with respect to p-xylene. Suitable purification methods include, for example, isomerization of mixed xylenes to increase the p-xylene concentration of the reaction mixture obtained from (III), rectification such as distillation, for example to separate o-xylene from the reaction mixture obtained from (III), selective adsorption, solvent removal, or selective precipitation.

### Step (IV)

Preferably the process, after (III), comprises
(IV) regenerating the molding.

There is no particular limitation as to the conditions for the regeneration of the molding. Preferably (IV) comprises heating the molding in an inert gas stream having a temperature which is preferably at least 10° C., more preferably at least 25° C., more preferably at least 50° C. higher than the temperature at which the contacting according to (III) is effected. For example, the inert gas stream used for regeneration is from 10 to 200° C., preferably from 25 to 150, more preferably from 50 to 100° C. higher than the temperature at which the contacting according to (III) is effected. As to the pressure of the regeneration process, it is preferred that the heating is effected at a pressure of the inert gas stream in the range of from 1 to 100 bar(abs), more preferably in the range of from 1.2 to 50 bar(abs), more preferably in the range of from 1.5 to 35 bar(abs). As to the composition of the inert gas stream there is no limitation as to the inert gas used in the regeneration step. Preferably, the inert gas stream comprises nitrogen and optionally oxygen.

The present invention is further illustrated by the following embodiments and combinations of embodiments as indicated by the respective dependencies and back-references. In particular, it is noted that in each instance where reference is made to more than two embodiments, for example in the context of a term such as “The process of any one of embodiments 1 to 4”, every embodiment in this range is meant to be explicitly disclosed, i.e. the wording of this term is to be understood as being synonymous to “The process of any one of embodiments 1, 2, 3, and 4”.
1. A process for preparing p-xylene, comprising

(I) providing a molding which comprises
(a) a zeolitic material,
(b) phosphorous,
(c) one or more metals M of the groups 3, 6, 10 to 14 of the periodic system of the elements, and
(d) a binder material;

(II) providing a gas stream comprising methanol;

(III) contacting the gas stream provided in (II) with the molding provided in (I), obtaining a reaction mixture comprising p-xylene.
2. The process of embodiment 1, wherein the gas stream provided in (II) additionally comprises one or more inert gases.
3. The process of embodiment 2, wherein the one or more inert gases are one or more of helium, neon, argon, nitrogen, carbon monoxide, and carbon dioxide, preferably one or more of argon, nitrogen, and carbon dioxide, wherein more preferably, the one or more inert gases comprise nitrogen, wherein more preferably, the one or more inert gases is nitrogen or a mixture of nitrogen and argon.
4. The process of embodiment 2 or 3, wherein at least 95 volume-%, preferably at least 98 volume-%, more preferably at least 99 volume-% of the gas stream provided in (II) consist of methanol and the one or more inert gases.
5. The process of any one of embodiments 1 to 4, wherein the gas stream provided in (II) comprises methanol in an amount in the range of from 30 to 70 volume-%, preferably in the range of from 40 to 60 volume-%, more preferably in the range of from 50 to 55 volume-%, based on the total volume of the gas stream.
6. The process of any one of embodiments 1 to 5, wherein the contacting according to (III) is effected at a temperature of the gas stream in the range of from 250 to 750° C., preferably in the range of from 300 to 700° C., more preferably in the range of from 350 to 650° C.
7. The process of any one of embodiments 1 to 6, wherein the contacting according to (III) is effected at a pressure of the gas stream in the range of from 1 to 100 bar(abs), preferably in the range of from to 1.2 to 50 bar(abs), more preferably in the range of from 1.5 to 35 bar(abs).
8. The process of any one of embodiments 1 to 7, wherein the contacting according to (III) is carried out in continuous mode.
9. The process of any one of embodiments 1 to 8, wherein the contacting according to (III) is carried out at a gas hourly space velocity (GHSV) in the range of from 500 to 3,000 h⁻¹, preferably in the range of from 1,000 to 2,500 h⁻¹, more preferably in the range of from 1,000 to 1,600 h⁻¹.
10. The process of any one of embodiments 1 to 9, wherein prior to (III), the molding provided according to (I) is heated in an inert gas, preferably to the temperature at which the contacting according to (III) is effected, said temperature being defined in embodiment 6.
11. The process of embodiment 10, wherein the inert gas comprises, preferably is the inert gas comprised in the gas stream provided in (II), said inert gas being defined in embodiment 3.
12. The process of any one of embodiments 1 to 11, wherein after (III), the process further comprises

(IV) regenerating the molding.
13. The process of embodiment 12, wherein (IV) comprises heating the molding in an inert gas stream having a temperature which is preferably at least 10° C., more preferably at least 25° C., more preferably at least 50° C. higher than the temperature at which the contacting according to (III) is effected.
14. The process of embodiment 13, wherein the heating is effected at a pressure of the inert gas stream in the range of from 1 to 100 bar(abs), preferably in the range of from 1.5 to 3 bar(abs).
15. The process of embodiment 13 or 14, wherein the inert gas stream comprises nitrogen and optionally oxygen.
16. The process of any one of embodiments 1 to 15, wherein the reaction mixture obtained from (III) comprises the p-xylene in a yield of at least 5.5%, preferably in a yield in the range of from 5.5 to 40%, more preferably in the range of from 8 to 35%, wherein the yield is the normalized yield calculated as disclosed in Reference Example 4.
17. The process of any one of embodiments 1 to 16, wherein the zeolitic material has a framework structure comprising YO₂ and X₂O₃ wherein Y is a tetravalent element and X is a trivalent element, wherein Y is preferably one or more of Si, Sn, Ti, Zr, and Ge, more preferably Si, and wherein X is preferably one or more of Al, B, In, and Ga, more preferably Al.
18. The process of embodiment 17, wherein in the zeolitic material, the molar ratio YO₂:X₂O₃ is in the range of from 10:1 to 100:1, preferably in the range of from 20:1 to 90:1, more preferably in the range of from 30:1 to 80:1, more preferably in the range of from 40:1 to 60:1, more preferably in the range of from 45:1 to 55:1.
19. The process of embodiment 17 or 18, wherein at least 95 weight-%, preferably at least 98 weight-%, more preferably at least 99 weight-%, more preferably at least 99.9 weight % of the framework structure of the zeolitic material consist of X, Y, O and H.
20. The process of any one of embodiments 1 to 19, wherein the zeolitic material has a framework structure of framework type ABW, ACO, AEI, AEL, AEN, AET, AFG, AFI, AFN, AFO, AFR, AFS, AFT, AFV, AFX, AFY, AHT, ANA, APC, APD, AST, ASV, ATN, ATO, ATS, ATT, ATV, AVL, AWO, AWW, BCT, BEA, BEC, BIK, BOF, BOG, BOZ, BPH, BRE, BSV, CAN, CAS, CDO, CFI, CGF, CGS, CHA, -CHI, -CLO, CON, CSV, CZP, DAC, DDR, DFO, DFT, DOH, DON, EAB, EDI, EEI, EMT, EON, EPI, ERI, ESV, ETR, EUO, *-EWT, EZT, FAR, FAU, FER, FRA, GIS, GIU, GME, GON, GOO, HEU, IFO, IFR, -IFU, IFW, IFY, IHW, IMF, IRN, IRR, -IRY, ISV, ITE, ITG, ITH, *-ITN, ITR, ITT, -ITV, ITW, IWR, IWS, IWV, IWW, JBW, JNT, JOZ, JRY, JSN, JSR, JST, JSW, KFI, LAU, LEV, LIO, -LIT, LOS, LOV, LTA, LTF, LTJ, LTL, LTN, MAR, MAZ, MEI, MEL, MEP, MER, MFI, MFS, MON, MOR, MOZ, *MRE, MSE, MSO, MTF, MTN, MTT, MTW, MVY, MWF, MWW, NAB, NAT, NES, NON, NPO, NPT, NSI, OBW, OFF, OKO, OSI, OSO, OWE, -PAR, PAU, PCR, PHI, PON, POS, PSI, PUN, RHO, -RON, RRO, RSN, RTE, RTH, RUT, RWR, RWY, SAF, SAO, SAS, SAT, SAV, SBE, SBN, SBS, SBT, SEW, SFE, SFF, SFG, SFH, SFN, SFO, SFS, *SFV, SFW, SGT, SIV, SOD, SOF, SOS, SSF, *-SSO, SSY, STF, STI, *STO, STT, STW, -SVR, SVV, SZR, TER, THO, TOL, TON, TSC, TUN, UEI, UFI, UOS, UOV, UOZ, USI, UTL, UWY, VET, VFI, VNI, VSV, WEI, -WEN, YUG, ZON, or a mixture of two or more of these framework types, or a mixed framework type of two or more of these framework types.
21. The process of any one of embodiments 1 to 20, wherein the zeolitic material has a framework structure of framework type BEA, MFI, MWW, MEL, MOR, MTT, MTW, FER, TOL, or TON, preferably of framework type MFI, MWW, MEL, or TON.
22. The process of any one of embodiments 1 to 21, wherein zeolitic material comprises, preferably is a ZSM-5 zeolitic material.
23. The process of any one of embodiments 1 to 21, wherein zeolitic material comprises, preferably is a ZBM-10 zeolitic material.
24. The process of any one of embodiments 1 to 21, wherein zeolitic material comprises, preferably is a ZSM-22 zeolitic material.
25. The process of any one of embodiments 1 to 21, wherein the zeolitic material comprises, preferably is, a ZSM-11 zeolitic material.
26. The process of any one of embodiments 1 to 21, wherein the zeolitic material comprises, preferably is, a ZBM-11 zeolitic material.
27. The process of any one of embodiments 1 to 26, wherein the molding comprises the phosphorous, calculated as elemental phosphorous, in an amount of at least 0.1 weight-%, preferably in an amount in the range of from 0.1 to 5 weight-%, more preferably in the range of from 0.1 to 2 weight-%, based on the total weight of the molding.
28. The process of any one of embodiments 1 to 27, wherein the one or more metals M are one or more of Ga, Zn, Ni, Mo, La and Pt, preferably one or more of Ga and Zn.
29. The process of any one of embodiments 1 to 28, wherein the molding comprises the one or more metals M, calculated as elemental M, in an amount of at least 1 weight-%, preferably in an amount in the range of from 1 to 4 weight-%, more preferably in the range of from 1.5 to 2.5 weight-%, based on the total weight of the molding, wherein said amount refers to the total amount of all metals M.
30. The process of any one of embodiments 1 to 29, wherein the one or more of metals M are comprised in the zeolitic material as extra-framework elements.
31. The process of any one of embodiments 1 to 30, wherein the binder material comprises, preferably is one or more of graphite, silica, titania, zirconia, alumina, and a mixed oxide of two or more of silicon, titanium, aluminum and zirconium, preferably one or more of graphite, silica, titania and zirconia, more preferably one or more of zirconia and silica.
32. The process of any one of embodiments 1 to 31, wherein in the molding, the weight ratio of the zeolitic material relative to the binder material is in the range of from 5:1 to 3:1, preferably in the range of from 4.5:1 to 3.5:1, more preferably the weight ratio is 4:1.
33. The process of any one of embodiments 1 to 32, wherein at least 95 weight-%, preferably at least 98 weight-%, more preferably at least 99 weight-%, more preferably at least 99.9 weight-% of the molding consist of the zeolitic material, the phosphorous, oxygen, the one or more metals M and the binder material.
34 The process of any one of embodiments 1 to 33, wherein the molding is a calcined molding, preferably calcined under a gas atmosphere having a temperature in the range of from 400 to 750° C., preferably in the range of from 450 to 650° C., more preferably in the range of from 500 to 550° C., wherein the gas atmosphere preferably comprises oxygen, the gas atmosphere more preferably is air.
35. The process of any one of embodiments 1 to 34, having a rectangular, a triangular, a hexagonal, a square, an oval or a circular cross section.
36. The process of any one of embodiments 1 to 35, being in the form of a star, a tablet, a sphere, or a cylinder.
37. The process of any one of embodiments 1 to 36, having a total pore area in the range of 20 to 80 m²/g, preferably in the range of from 25 to 50 m²/g, more preferably in the range of 30 to 40 m²/g determined as described in Reference Example 1 herein.
38. The process of any one of embodiments 1 to 37, having a total pore area of 35 m²/g determined as described in Reference Example 1 herein.
39. The process of any one of embodiments 1 to 38, having a BET specific surface area in the range of from 100 to 500 m²/g, preferably in the range of from 100 to 350 m²/g, determined as described in Reference Example 2 herein.
40. The process of any one of embodiments 1 to 39, having a total intrusion volume in the range of from 0.15 to 3 mL/g, preferably in the range of from 0.2 to 2.5 mL/g, more preferably in the range of from 0.5 to 0.85 mL/g, determined as described in Reference Example 3 herein.
41. The process of any one of embodiments 1 to 40, wherein providing a molding according to (I) comprises preparing a molding as defined in any one of embodiments 17 to 40, wherein preparing the molding preferably comprises
   (i) providing the zeolitic material;
   (ii) mixing the zeolitic material provided in (i) with a source of the binder material;
   (iii) subjecting the mixture obtained from (ii) to molding;
   (iv) impregnating the molding obtained from (iii) with a source of the one or more metals M and a source of the phosphorous.
42. The process of embodiment 41, The process of embodiment 26, wherein the binder material is silica and the source of the binder material comprises one or more of a colloidal silica, a silica gel and a waterglass, preferably a colloidal silica.
43. The process of embodiment 42, wherein according to (ii), the zeolitic material provided in (i) is mixed with the source of the binder material and a kneading agent, wherein the kneading agent is preferably a polar protic kneading agent, more preferably one or more of water, an alcohol, and a mixture of two or more thereof, more preferably one or more of water, a C1-C5 alcohol, and a mixture of two or more thereof, more preferably one or more of water, a C1-C4 alcohol, and a mixture of two or more thereof, more preferably one or more of water, methanol, ethanol, propanol, and a mixture of two or more thereof, wherein more preferably, the kneading agent comprises, more preferably is water.
44. The process of embodiment 43, wherein in the mixture obtained from (ii), the weight ratio of the kneading agent relative to the zeolitic material is in the range of from 0.5:1 to 2:1, preferably in the range of from 0.75:1 to 1.7:1, more preferably in the range of from 1.0:1 to 1.5:1.
45. The process of any one of embodiments 41 to 44, wherein according to (ii), the zeolitic material provided in (i) is mixed with the source of the binder material and a mesopore forming agent and preferably the kneading agent.
46. The process of embodiment 45, wherein the mesopore forming agent is one or more of a polymer, a carbohydrate, graphite, and a mixture of two or more thereof, preferably one or more of a polymeric vinyl compound, a polyalkylene oxide, a polyacrylate, a polyolefin, a polyamide, a polyester, a cellulose, a cellulose derivative, a sugar, and a mixture of two or more thereof, more preferably one or more of a polystyrene, a polyethylene oxide, a polypropylene oxide, a cellulose derivative, a sugar, and a mixture of two or more thereof, more preferably one or more of a polystyrene, a polyethylene oxide, a C1-C2 hydroxyalkylated cellulose derivative, a C1-C2 alkylated cellulose derivative, a sugar, and a mixture of two or more thereof, more preferably one or more of a polystyrene, a polyethylene oxide, a hydroxyethyl methyl cellulose, and a mixture of two or more thereof, wherein more preferably, the mesopore forming agent comprises, more preferably is, one or more of a polyethylene oxide and a hydroxyethyl methyl cellulose.
47. The process of embodiment 45 or 46, wherein in the mixture obtained from (ii), the weight ratio of the mesopore forming agent relative to the zeolitic material is in the range of from 0.001:1 to 0.3:1, preferably in the range of from 0.005:1 to 0.1:1, more preferably in the range of from 0.01:1 to 0.05:1, more preferably in the range of from 0.02:1 to 0.04:1, more preferably in the range of from 0.025:1 to 0.035:1.
48. The process of any one of embodiments 41 to 47, wherein the mixing according to (ii) comprises kneading.
49. The process of any one of embodiments 41 to 48, wherein subjecting to molding according to (iii) comprises extruding the mixture obtained from (ii).
50. The process of any one of embodiments 41 to 49, wherein after (iii) and prior to (iv), the molding obtained from (ii) is subjected to drying.
51. The process of embodiment 50, wherein the drying is carried out in a gas atmosphere having a temperature in the range of from 50 to 200° C., preferably in the range of from 75 to 150° C., more preferably in the range of from 100 to 125° C.
52. The process of any one of embodiments 41 to 51, wherein the molding obtained from (iii), preferably the molding obtained from drying according to embodiment 50 or 51, is subjected to calcining.
53. The process of embodiment 52, wherein the calcining is carried out in a gas atmosphere having a temperature in the range of from 400 to 750° C., preferably in the range of from 450 to 650° C., more preferably in the range of from 500 to 550° C., wherein the gas atmosphere preferably comprises oxygen, the gas atmosphere more preferably is air.
54. The process of any one of embodiments 41 to 53, wherein the impregnating according to (iv) comprises spray-impregnating the molding with the source of the one or more metals M and the source of the phosphorous.

55. The process of embodiment 54, wherein (iv) further comprises preparing a solution comprising the source of the one or more metals M and the source of the phosphorous, or a solution comprising the source of the one or more metals M and a solution comprising the source of the phosphorous.
56. The process of embodiment 55, wherein preparing the solution or the solutions comprises dissolving the source of the one or more metals M and/or the source of the phosphorous in water, preferably deionized water.
57. The process of embodiment 55 or 56, wherein the solution or the solutions is or are sprayed onto the molding through a nozzle, preferably a glass nozzle.
58. The process of any one of embodiments 41 to 57, wherein, the molding obtained from (iv) is subjected to drying.
59. The process of embodiment 58, wherein the drying is carried out in a gas atmosphere having a temperature in the range of from 50 to 200° C., preferably in the range of from 75 to 150° C., more preferably in the range of from 100 to 125° C.
60. The process of any one of embodiments 41 to 59, wherein the molding obtained from (iv), preferably the molding obtained from drying according to embodiment 58 or 59, is subjected to calcining.
61. The process of embodiment 60, wherein the calcining is carried out in a gas atmosphere having a temperature in the range of from 400 to 750° C., preferably in the range of from 450 to 650° C., more preferably in the range of from 500 to 550° C., wherein the gas atmosphere preferably comprises oxygen, the gas atmosphere more preferably is air.
62. The process of any one of embodiments 41 to 61, wherein (iv) comprises
   (iv-1) impregnating the molding with the source of the one or more metals M;
   (iv-2) impregnating the molding obtained from (iv-1) with the source of phosphorous.
63. The process of embodiment 62, wherein the impregnating according to (iv-1) comprises spray-impregnating the molding with the source of the one or more metals M.
64. The process of embodiment 63, wherein (iv-1) further comprises preparing a solution comprising the source of the one or more metals M.
65. The process of embodiment 64, wherein preparing the solution comprises dissolving the source of the one or more metals M in water, preferably deionized water.
66. The process of embodiment 64 or 65, wherein the solution is sprayed onto the molding through a nozzle, preferably a glass nozzle.
67. The process of any of embodiments 62 to 66, wherein the impregnating according to (iv-2) comprises spray-impregnating the molding with the source of the phosphorous.
68. The process of embodiment 67, wherein (iv-2) further comprises preparing a solution comprising the source of the phosphorous.
69. The process of embodiment 68, wherein preparing the solution comprises dissolving the source of the phosphorous in water, preferably deionized water.
70. The process of embodiment 68 or 69, wherein the solution is sprayed onto the molding through a nozzle, preferably a glass nozzle.
71. The process of any one of embodiments 62 to 70, wherein after (iv-1) and prior to (iv-2), the molding obtained from (iv-1) is subjected to drying.
72. The process of embodiment 71, wherein the drying is carried out in a gas atmosphere having a temperature in the range of from 50 to 200° C., preferably in the range of from 75 to 150° C., more preferably in the range of from 100 to 125° C.
73. The process of any one of embodiments 62 to 72, wherein the molding obtained from (iv-2), preferably the molding obtained from drying according to embodiment 71 or 72, is subjected to calcining.
74. The process of embodiment 73, wherein the calcining is carried out in a gas atmosphere having a temperature in the range of from 400 to 750° C., preferably in the range of from 450 to 650° C., more preferably in the range of from 500 to 550° C. wherein the gas atmosphere preferably comprises oxygen, the gas atmosphere more preferably is air.
75. The process of any one of embodiments 41 to 61, wherein (iv) comprises
   (iv-1′) impregnating the molding with the source of the phosphorous;
   (iv-2′) impregnating the molding obtained from (iv-1′) with the source of the one or more metals M.
76. The process of embodiment 75, wherein the impregnating according to (iv-1′) comprises spray-impregnating the molding with the source of phosphorous.
77. The process of embodiment 76, wherein (iv-1′) further comprises preparing a solution comprising the source of the phosphorous.
78. The process of embodiment 77, wherein preparing the solution or the solutions comprises dissolving the source of the phosphorous in water, preferably deionized water.
79. The process of embodiment 77 or 78, wherein the solution is sprayed onto the molding through a nozzle, preferably a glass nozzle.
80. The process of any of embodiments 75 to 79, wherein the impregnating according to (iv-2′) comprises spray-impregnating the molding with the source of the one or more metals M.
81. The process of embodiment 80, wherein (iv-2′) further comprises preparing a solution comprising the source of the one or more metals M.
82. The process of embodiment 82, wherein preparing the solution dissolving the source of the one or more metals M in water, preferably deionized water.
83. The process of embodiment 81 or 82, wherein the solution is sprayed onto the molding through a nozzle, preferably a glass nozzle.
84. The process of any one of embodiments 75 to 83, wherein after (iv-1′) and prior to (iv-2′), the molding obtained from (iv-1′) is subjected to drying.
85. The process of embodiment 84, wherein the drying is carried out in a gas atmosphere having a temperature in the range of from 50 to 200° C., preferably in the range of from 75 to 150° C., more preferably in the range of from 100 to 125° C.
86. The process of any one of embodiments 75 to 85, wherein after (iv-2′), preferably after the drying according to embodiment 69 or 70, the molding obtained is subjected to calcining.
87. The process of embodiment 86, wherein the calcining is carried out in a gas atmosphere having a temperature in the range of from 400 to 750° C., preferably in the range of from 450 to 650° C., more preferably in the range of from 500 to 550° C. wherein the gas atmosphere preferably comprises oxygen, the gas atmosphere more preferably is air.
88. The process of any one of embodiments 41 to 87, wherein the source of the one or more metals M is a salt of said one or more metals M, preferably an inorganic salt of said one or more metals M, more preferably a bromide, a chlorate, a chloride, an iodide, a nitrate, or a sulfate of said one or more metals M, wherein more preferably, the source of the one or more metals M is a nitrate.
89. The process of any one of embodiments 41 to 88, wherein the one or more metals M comprise Zn and the source of Zn is zinc(II) nitrate.
90. The process of any one of embodiments 41 to 89, wherein the one or more metals M comprise Ga and the source of Ga is gallium(III) nitrate.
91. The process of any one of embodiments 41 to 90, wherein the source of the phosphorous is one or more of phosphorous acid (H₃PO₃), phosphoric acid (H₃PO₄), a salt of phosphorous acid, a salt of phosphoric acid, and a dihydrogen phosphate anion containing compound, wherein the dihydrogen phosphate anion containing compound is preferably one or more of monoammonium phosphate and diammonium phosphate, wherein the source of the phosphorous is more preferably one or more of phosphorous acid (H₃PO₃) and phosphoric acid (H₃PO₄), more preferably is phosphoric acid.

The present invention is further illustrated by the following embodiments and combinations of embodiments as indicated by the respective dependencies and back-references. In particular, it is noted that in each instance where reference is made to more than two embodiments, for example in the context of a term such as “The molding of any one of embodiments 1 to 4”, every embodiment in this range is meant to be explicitly disclosed, i.e. the wording of this term is to be understood as being synonymous to “The molding of any one of embodiments 1, 2, 3, and 4”.
1. A molding comprising
   (a) a zeolitic material;
   (b) phosphorous;
   (c) one or more metals M of groups 3, 6 and 10 to 14 of the periodic system of the elements;
   (d) a binder material.
2. The molding of embodiment 1, wherein the zeolitic material has a framework structure comprising YO₂ and X₂O₃ wherein Y is a tetravalent element and X is a trivalent element, wherein Y is preferably one or more of Si, Sn, Ti, Zr, and Ge, more preferably Si, and wherein X is preferably one or more of Al, B, In, and Ga, more preferably Al.
3. The molding of embodiment 2, wherein in the zeolitic material, the molar ratio YO₂:X₂O₃ is in the range of from 10:1 to 100:1, preferably in the range of from 20:1 to 90:1, more preferably in the range of from 30:1 to 80:1, more preferably in the range of from 40:1 to 60:1, more preferably in the range of from 45:1 to 55:1.
4. The molding of embodiment 2 or 3, wherein at least 95 weight-%, preferably at least 98 weight-%, more preferably at least 99 weight-%, more preferably at least 99.9 weight-% of the framework structure of the zeolitic material consist of X, Y, O and H.
5. The molding of any one of embodiments 1 to 4, wherein the zeolitic material has a framework structure of framework type ABW, ACO, AEI, AEL, AEN, AET, AFG, AFI, AFN, AFO, AFR, AFS, AFT, AFV, AFX, AFY, AHT, ANA, APC, APD, AST, ASV, ATN, ATO, ATS, ATT, ATV, AVL, AWO, AWW, BCT, BEA, BEC, BIK, BOF, BOG, BOZ, BPH, BRE, BSV, CAN, CAS, CDO, CFI, CGF, CGS, CHA, -CHI, -CLO, CON, CSV, CZP, DAC, DDR, DFO, DFT, DOH, DON, EAB, EDI, EEI, EMT, EON, EPI, ERI, ESV, ETR, EUO, *-EWT, EZT,

FAR, FAU, FER, FRA, GIS, GIU, GME, GON, GOO, HEU, IFO, IFR, -IFU, IFW, IFY, IHW, IMF, IRN, IRR, -IRY, ISV, ITE, ITG, ITH, *-ITN, ITR, ITT, -ITV, ITW, IWR, IWS, IWV, IWW, JBW, JNT, JOZ, JRY, JSN, JSR, JST, JSW, KFI, LAU, LEV, LIO, -LIT, LOS, LOV, LTA, LTF, LTJ, LTL, LTN, MAR, MAZ, MEI, MEL, MEP, MER, MFI, MFS, MON, MOR, MOZ, *MRE, MSE, MSO, MTF, MTN, MTT, MTW, MW, MWF, MWW, NAB, NAT, NES, NON, NPO, NPT, NSI, OBW, OFF, OKO, OSI, OSO, OWE, -PAR, PAU, PCR, PHI, PON, POS, PSI, PUN, RHO, -RON, RRO, RSN, RTE, RTH, RUT, RWR, RWY, SAF, SAO, SAS, SAT, SAV, SBE, SBN, SBS, SBT, SEW, SFE, SFF, SFG, SFH, SFN, SFO, SFS, *SFV, SFW, SGT, SIV, SOD, SOF, SOS, SSF, *-SSO, SSY, STF, STI, *STO, STT, STW, -SVR, SW, SZR, TER, THO, TOL, TON, TSC, TUN, UEI, UFI, UOS, UOV, UOZ, USI, UTL, UWY, VET, VFI, VNI, VSV, WEI, -WEN, YUG, ZON, or a mixture of two or more of these framework types, or a mixed framework type of two or more of these framework types.
6. The molding of any one of embodiments 1 to 5, wherein the zeolitic material has a framework structure of framework type BEA, MFI, MWW, MEL, MOR, MTT, MTW, FER, TOL, or TON, preferably of framework type MFI, MWW, MEL, or TON.
7. The molding of any one of embodiments 1 to 6, wherein zeolitic material comprises, preferably is a ZSM-5 zeolitic material.
8. The molding of any one of embodiments 1 to 6, wherein zeolitic material comprises, preferably is a ZBM-10 zeolitic material.
9. The molding of any one of embodiments 1 to 6, wherein zeolitic material comprises, preferably is a ZSM-22 zeolitic material.
10. The molding of any one of embodiments 1 to 6, wherein the zeolitic material comprises, preferably is, a ZSM-11 zeolitic material.
11. The molding of any one of embodiments 1 to 6, wherein the zeolitic material comprises, preferably is, a ZBM-11 zeolitic material.
12. The molding of any one of embodiments 1 to 11, comprising the phosphorous, calculated as elemental phosphorous, in an amount of at least 0.1 weight-%, preferably in an amount in the range of from 0.1 to 5 weight-%, more preferably in the range of from 0.1 to 2 weight-%, based on the total weight of the molding.
13. The molding of any one of embodiments 1 to 12, wherein the one or more metals M are one or more of Ga, Zn, Ni, Mo, La and Pt, preferably one or more of Ga and Zn.
14. The molding of any one of embodiments 1 to 13, comprising the one or more metals M, calculated as elemental M, in an amount of at least 1 weight-%, preferably in an amount in the range of from 1 to 4 weight-%, more preferably in the range of from 1.5 to 2.5 weight-%, based on the total weight of the molding, wherein said amount refers to the total amount of all metals M.
15. The molding of any one of embodiments 1 to 14, wherein the one or more of metals M are comprised in the zeolitic material as extra-framework elements.
16. The molding of any one of embodiments 1 to 15, wherein the binder material comprises, preferably is one or more of graphite, silica, titania, zirconia, alumina, and a mixed oxide of two or more of silicon, titanium, aluminum and zirconium, preferably one or more of graphite, silica, titania and zirconia, more preferably one or more of zirconia and silica.
17. The molding of any one of embodiments 1 to 16, wherein in the molding, the weight ratio of the zeolitic material relative to the binder material is in the range of from 5:1 to 3:1, preferably in the range of from 4.5:1 to 3.5:1, wherein more preferably, the weight ratio is 4:1.
18. The molding of any one of embodiments 1 to 17, wherein at least 95 weight-%, preferably at least 98 weight-%, more preferably at least 99 weight-%, more preferably at least 99.9 weight-% of molding consist of the zeolitic material, the phosphorous, oxygen, the metal M and the binder material.
19. The molding of any one of embodiments 1 to 18, being a calcined molding, preferably calcined under a gas atmosphere having a temperature the range of from 400 to 750° C., more preferably in the range of from 450 to 650° C., more preferably in the range of from 500 to 550° C., wherein the gas atmosphere preferably comprises oxygen, the gas atmosphere more preferably is air.
20. The molding of any one of embodiments 1 to 19, having a rectangular, a triangular, a hexagonal, a square, an oval or a circular cross section.
21. The molding of any one of embodiments 1 to 20, being in the form of a star, a tablet, a sphere, or a cylinder.
22. The molding of any one of embodiments 1 to 21, having a total pore area in the range of 20 to 80 m²/g, preferably in the range of from 25 to 50 m²/g, more preferably in the range of 30 to 40 m²/g determined as described in Reference Example 1 herein.
23. The molding of any one of embodiments 1 to 22, having a total pore area of 35 m²/g determined as described in Reference Example 1 herein.
24. The molding of any one of embodiments 1 to 23, having a BET specific surface area in the range of from 100 to 500 m²/g, preferably in the range of from 100 to 350 m²/g, determined as described in Reference Example 2 herein.
25. The molding of any one of embodiments 1 to 24, having a total intrusion volume in the range of from 0.15 to 3 mL/g, preferably in the range of from 0.2 to 2.5 mL/g, more preferably in the range of from 0.5 to 0.85 mL/g, determined as described in Reference Example 3 herein.

The invention is further illustrated by the following reference examples, examples, and comparative examples.

### EXAMPLES

### Reference Example 1

### Measurement of the Total Pore Area

The total pore area was determined according to the method disclosed in DIN 66134:1998-02 “Determination of the pore size distribution and the specific surface area of mesoporous solids by means of nitrogen sorption” issued in February 1998.

### Reference Example 2

### Measurement of the BET Specific Surface Area

The BET specific surface area was determined according to the method disclosed in DIN ISO 9277:2010 “Determination of the specific surface area of solids by gas absorption” as issued on January 2014.

### Reference Example 3

### Measurement of the Total Intrusion Volume

The total pore area was determined by mercury intrusion in accordance with the method disclosed in DIN 66133 as issued in 1993.

### Reference Example 4

### Determination of the Yield

The yield of p-xylene is the normalized yield and is calculated as follows:

Y_{product} [%]⁼(RC_{product}[g(C)/h]/SUM_{RC}[g(C)/h])*100

SUM_{RC}=(RC_{productFID})+(RC_{CO-TCD})+(RC_{CO2-TCD})

Normalized yield factor=100/Sum_{yields}

Y_{product-norm}[%]=Y_{product}*normalized yield factor
wherein

RC=rate of carbon, in gram per carbon per hour, g(C)/h
Y_{product}=yield of the product
Y_{product-norm}=normalized yield of the product
Sum_{RC}=sum of the rates of carbon
Sum_{yield}=sum of the yields
RC_{productFID}=rate of the carbon measured with the flame ionization (FID) method
RC_{CO-TCD}=rate of the carbon of CO measured with the thermal conductivity (TCD) detector
RC_{CO2-TCD}=rate of the carbon of CO₂ measured with the thermal conductivity (TCD) detector

The products and methanol feed are detected at flame ionization (FID) detector, whereas CO and CO₂ at a thermal conductivity (TCD) detector. FID and TCD are given next to the carbon rates. All yields are then normalized to 100.

### Comparative Example 1

### Preparation of a Molding Comprising a ZSM-5 Zeolitic Material Impregnated with Zn by Extrusion

a) Spray-Impregnation of ZSM-5 with Zn

### Starting Material

| | | |
|---|---|---|
| ZSM-5 zeolitic material | 170 | g |
| Deionized water (DI water) | 110 | g |
| Zn(NO₃)₂ × 6H₂O | 9.3 | g |

For impregnation, 170 g of ZS-5 were introduced into a round bottom flask and placed in a rotary evaporator. The Zn(NO₃)₂×6 H₂O were dissolved in deionized water. The metal nitrate solution was introduced into a dropping funnel, and sprayed gradually onto the extrudates through a glass spray nozzle flooded with 100 l/h of N₂ while rotating. On completion of addition of the metal nitrate solution, the zeolitic material was rotated further for 10 min. The impregnate zeolitic material were dried in air at 120° C. for 4h and calcined in air at 500° C. for 5h. Afterwards, the obtained powder was removed and dried in a forced air drying oven for 4 h at 120° C. and then calcined in the muffle furnace for 5 h at 500° C. (heating rate 2 K/min) under air.

The obtained material had a BET specific surface area of 392 m²/g, a total intrusion volume 1.5991 mL/g and a total pore area at 68.693 m²/g. Elemental analysis of the obtained material: H <0.01 weight-%, Al 1.80 weight-%, Na <0.01 weight-%, Zn 1.1 weight-%, Si 43 weight-%. Elemental analysis of the starting material ZSM-5: H 0.02 weight-%, Al 1.80 weight-%, Na<0.01 weight-%, Zn<0.01 weight-%, Si 44 weight-%.

### b) Preparation of a Molding by Extrusion

### Starting Materials:

| | | |
|---|---|---|
| Zeolitic material of a) | 140 | g |
| Ludox ® AS-40 (colloidal silica, 40 weight-% | 87.5 | g |
| Walocel ® (hydroxyethyl methyl cellulose) | 7 | g |
| DI water (deionized water) | 73 | g |

The zeolitic material of a) was placed in a kneader, Walocel was added and pre-mixed for 5 min. Ludox was then added and the mixture was kneaded for 5 min. Thereafter 3 g of DI water were added and the material was kneaded for 15 min. Thereafter, the kneaded material was molded via an extrusion press (forming pressure: 120-150 bar(abs)) leading to strands having a diameter of 2.5 mm. The resulting strands were placed in a porcelain bowl in a drying oven at 120° C. for 4 h under air and then calcined in a muffle furnace at 500° C. (heating rate: 2 K/min) for 5 h under air. 168.31 g material were obtained, having a bulk density of 0.492 g/cm³. The obtained material had a BET specific surface area of 340 m²/g, a total intrusion volume of 0.5208 mL/g and a total pore area of 74.465 m²/g. Elemental analysis of the material: H 0.01 weight-%, Al 1.5 weight-%, Na 0.04 weight-%, Si 43 weight-%, Zn 0.91 weight-%.

### Example 1

### Preparation of a Molding Comprising a ZSM-22 Zeolitic Material by Impregnation with Ga and P

### a) Preparation of a ZSM-22 Zeolitic Material

### Starting Materials:

### Solution 1:

| | |
|---|---|
| Hexamethylendiamine 70% in water | 406 g |
| Aerosil ® 200 | 185 g |
| DI water | 700 g |

### Solution 2

| | | |
|---|---|---|
| Al₂(SO₄)₃ × 18 H₂O (Aldrich 11044-2, | 20.2 | g |
| 5 kg, Lot. #SZBD1200V) | | |
| DI Water | 270 | g |

### Solution 1

Hexamethylendiamine was placed in a beaker of 2 I volume. DI water was added and the solution was stirred for 5 min at room temperature. Aerosil was added under stirring conditions. The stirring was continued for 2 h at room temperature. The pH of the obtained solution was 12.6.

### Solution 2

The DI water was added under stirring to Al₂(SO₄)₃×18 H₂O.

Solution 1 was charged into an autoclave under stirring at 100 rpm and heated to 70° C. Solution 2 was then added under stirring at 220 rpm. The stirring was continued for 5 min. The stirring speed was then reduced to 100 rpm, the solution was kept under stirring at 70° C. under a constant pressure for 4 h. The solution was then heated to 150° C. under a constant pressure with stirring for 170 h. The pressure used was 3.6 bar(abs). Thereafter the suspension having pH of 12.0 was filtered off by means of a porcelain filter (blue band filter). The filter cake was washed three times with 1000 ml of DI water and dried in a forced-air drying oven at 120° C. for 4 h and then in a muffle furnace for 5 hours at 500° C. (heating rate 2 K/min) under air. 143.82 g material were obtained. The material had a BET specific surface area of 201 m²/g, a total intrusion volume at 5.3432 mL/g and a total pore area of 73.381 m²/g. Elemental analysis of the material: H 0.44 weight-%, Al 1.0 weight-%, Si 44 weight-%.

### b) Preparation of the Molding

### Starting Material

| | | |
|---|---|---|
| Zeolitic material of a) | 120 | g |
| Ludox ® AS 40 | 75 | g |
| Walocel ® | 6 | g |
| DI water | 180 | g |
| polyethylene oxide (PEO) (Alkox E-160) | 3.6 | g |

120 g of the zeolitic material of a) were placed in a kneader, Walocel® was added and pre-mixed for 5 min. Ludox® was then added and the mixture was kneaded for 5 min. Thereafter 150 g of DI were added and compacted within 15 minutes. PEO was then added and the mixture was kneaded for 5 min. 30 g of DI water were then added and the mixture was kneaded for 20 min. Thereafter, the kneaded material was formed (2.5 mm) via an extrusion press (forming pressure: 95-150 bar). The resulting string were placed in a porcelain bowl in a drying oven at 120° C. for 4 h and dried and then calcined in a muffle furnace at 500° C. (heating rate: 2 K/min) for 5 h under air. 142.01 g material were obtained, having a bulk density of 0.310 g/cm³. The material had a BET specific surface area of 196 m² /g, a total intrusion volume of 1.1770 mL/g and a total pore area of 77.861 m²/g. Elemental analysis of the material: H 0.22 weight-%, Al 0.84 weight-%, Si 45 weight-%.

c) Spray-Impregnation with Ga of the Molding

For impregnation, the 30 g of the molding of b) were introduced into a round bottom flask and placed in a rotary evaporator. 5.5 g of Ga(NO₃)₃×7 H₂O were dissolved in 15 g of DI water. The metal nitrate solution was introduced into a dropping funnel, and sprayed gradually onto the extrudates through a glass spray nozzle flooded with 100 l/h of N₂ while rotating. On completion of addition of the metal nitrate solution, the molding were rotated further for 10 min. Afterwards, the strands were removed and dried in a forced air drying oven for 4 h at 120° C. and then calcined in a muffle furnace for 5 h at 500° C. (heating rate 2 K/min) under air. 31.41 g material were obtained.

d) Spray-Impregnation with P of the Molding of c) to Prepare

For impregnation, 15 g of the molding of c) were introduced into a round bottom flask and placed in a rotary evaporator. 0.6 g of H₃PO₄ were dissolved in 8 g of DI water (phosphorows solution). The phosphorous solution was introduced into a dropping funnel, and sprayed gradually onto the molding through a glass spray nozzle flooded with 100 l/h of N2 while rotating. On completion of addition of the phosphorous solution, the extrudates were rotated further for 10 min. Afterwards, the strands were removed and dried in the forced air drying oven for 4 h at 120° C. and then calcined in a muffle furnace for 5 h at 500° C. (heating rate 2 K/min) under air.

The material had a BET specific surface area of 196 m²/g, a total intrusion volume of 1.0834 mL/g and a total pore area of 66,669 m²/g. Elemental analysis of the material: H 0.03 weight-%, Al 0.80 weight-%, Ga 3.0 weight-%, P 0.13 weight-%, Si 43 weight-%.

### Example 2

### Synthesis of a Molding Comprising the Zeolitic Material ZBM-10 Comprising Impregnation with Ga and P

### a) Preparation of a ZBM-10 Zeolitic Material

### Starting Materials:

### Solution 1

| | |
|---|---|
| Hexamethylendiamine 70% in water | 456 g |
| Aerosil ® 200 | 185 g |
| DI water | 700 g |

### Solution 2

| | | |
|---|---|---|
| Al₂(SO₄)₃ × 18 H₂O (Aldrich 11044-2, | 20.2 | g |
| 5 kg, Lot. #SZBD1200V) | | |
| DI water | 270 | g |

### Solution 1

Hexamethylendiamine was placed in a beaker of 2 l volume. Water was added and the solution was stirred for 5 min at room temperature. Aerosil was added under stirring conditions. The stirring was continued for 2 h at room temperature. The pH of the solution was 12.88.

### Solution 2

The DI water was added under stirring to Al₂(SO₄)₃×18 H₂O.

Solution 1 was charged into an autoclave with stirring at 200 rpm and heated to 70° C. Solution 2 was then added under stirring at 220 rpm. The stirring was continued for 5 min. The solution was kept under stirring at 70° C. under a constant pressure for 4 h. The solution was then heated to 150° C. under a constant pressure under stirring for 170 h. Afterwards, the suspension having a pH of 12.31 was filtered off by means of a porcelain filter (blue band filter). The filter cake was washed three times with 1000 ml of DI water and dried in a forced-air drying oven at 120° C. for 4 h and then calcined in a muffle furnace for 5 h at 500° C. (heating rate 2 K/min) under air. 187.75 g material were obtained. The material had a BET specific surface area of 347 m² /g. Elemental analysis of the material: H 0.14 weight-%, Al 0.91 weight-%, Si 44 weight-%.

### b) Preparation of the Molding

### Starting Materials:

| | | |
|---|---|---|
| Zeolitic material of a) | 100 | g |
| Ludox ® AS 40 | 62.5 | g |
| Walocel ® | 6 | g |
| DI water | 75 | g |
| polyethylene oxide (PEO) (Alkox E-160) | 3 | g |

100 g of the zeolitic material of a) were placed in the kneader, Walocel was added and pre-mixed for 5 min. Ludox was then added and the mixture was kneaded for 5 min. Thereafter 50 g of DI water were added and the mixture was compacted within 15 min. 3.6 g of PEO were then added and the mixture was kneaded for 5 min. 25 g of DI water were then added and the mixture was kneaded for 5 min. Thereafter, the kneaded material was molded with an extrusion press (2.5 mm; forming pressure: 95-150 bar). The resulting strands were placed in a porcelain bowl in a drying oven at 120° C. for 4 h and dried and then calcined in a muffle furnace at 500° C. (heating rate: 2 K/min) for 5 h under air. 114.44 g material were obtained, having a bulk density of 0.443 g/cm³. The material had a BET surface area of 310 m² /g, a total intrusion volume of 0.6432 mL/g and a total pore area of 37.627 m²/g. Elemental analysis of the material: H 0.03 weight-%, Al 0.72 weight-%, Si 45 weight-%.

c) Spray-Impregnation with Ga of the Molding of b)

For impregnation, 30 g of the molding of b) were introduced into a round bottom flask and placed in a rotary evaporator. 5.5 g of Ga(NO₃)₃×7 H₂O were dissolved in 15 g of DI water. The metal nitrate solution was introduced into a dropping funnel, and sprayed gradually onto the extrudates through a glass spray nozzle flooded with 100 l/h of N₂ while rotating. On completion of addition of the metal nitrate solution, the molding were rotated further for 10 min.

Afterwards, they strands were removed and dried in a forced air drying oven 4 h at 120° C. and then calcined in a muffle furnace for 5 h at 500° C. (heating rate 2 K/min) under air. 31.20 g material were obtained.

d) Spray-Impregnation with P of the Molding of c) to Prepare the Molding of the Title

For impregnation, 16.15 g of the molding of c) were introduced into a round bottom flask and placed in a rotary evaporator. 0.66 g of H₃PO₄ were dissolved in 8 g of DI water (phosphorous solution). The phosphorous solution was introduced into a dropping funnel, and sprayed gradually onto the molding through a glass spray nozzle flooded with 100 l/h of N₂ while rotating. On completion of addition of the phosphorous solution, the molding was rotated further for 10 min.

Afterwards, the impregnated strands were removed and dried in a forced air drying oven for 4 h at 120° C. and then calcined in a muffle furnace for 5 h at 500° C. (heating rate 2 K/min) under air. 16.28 g material were obtained. The material had a BET specific surface area of 300 m²/g. Elemental analysis of the material: H 0.01 weight-%, Al 0.69 weight-%, Ga 2.7 weight-%, P 1.1 weight-%, Si 42 weight-%.

### Example 3

### Preparation of a Molding Comprising a ZBM-10 Zeolitic Material Comprising Impregnation with Zn and P

### a) Preparation of a ZBM-10 Zeolitic Material

A ZBM-10 zeolitic material was provided, prepared as described in Example 2 a) above.

### b) Preparation of the Molding

A molding comprising the ZBM-10 zeolitic material of a) was prepared as described in Example 2 b) above.

c) Spray-Impregnation with Zn of the Molding of b)

For impregnation, 30 g of the molding of b) were introduced into a round bottom flask and placed in a rotary evaporator. 3.2 g of Zn(OAc)₂×2 H₂O were dissolved in 15 g of DI water. The metal nitrate solution was introduced into a dropping funnel, and sprayed gradually for 5 min onto the molding through a glass spray nozzle flooded with 100 l/h of N₂ while rotating. On completion of addition of the metal nitrate solution, the moldings were rotated further for 15 min.

Afterwards, they strands were removed and dried in a forced air drying oven 4 h at 120° C. and then calcined in a muffle furnace for 5 h at 500° C. (heating rate 2 K/min) under air. 11.18 g material were obtained.

d) Spray-impregnation with P of the molding of c) to prepare the molding of the title

For impregnation, 30 g of the molding of c) were introduced into a round bottom flask and placed in a rotary evaporator. 0.66 g of H₃PO₄ were dissolved in 10 g of DI water (phosphorous solution). The phosphorous solution was introduced into a dropping funnel, and sprayed gradually onto the molding through a glass spray nozzle flooded with 100 l/h of N₂ while rotating. On completion of addition of the phosphorous solution, the molding was rotated further for 10 min. Afterwards, the strands were removed and dried in a forced air drying oven at 120° C. and then calcined in a muffle furnace for 5 h at 500° C. (heating rate 2 K/min) under air.

The material had a BET specific surface area of 277 m²/g. Elemental analysis of the material: H 0.01 weight-%, Al 0.69 weight-%, Zn 2.8 weight-%, P 1.0 weight-%, Si 43 weight-%.

### Example 4

### General Procedure for Preparing P-Xylene from Methanol

### a) Start-Up

The catalyst (molding) was heated in a gas stream (nitrogen 90 volume-%, Argon 10 volume-%) to the reaction temperature followed by a dwell time of 2 h.

### b) Reaction

0.5 ml of catalyst were loaded into a fixed bed reactor. The catalyst of a) was exposed to multiple reaction/regeneration cycles. In the first (MTX1), second (MTX2) and third (MTX3) reaction cycle, the reaction temperature was set to 450° C. and the reactor pressure at the outlet to 5 bar(abs). The gaseous hourly space velocity (GHSV) was 1,000 h⁻¹. The volume ratios of the individual gases at the reactor inlet were MeOH/Ar/N₂=52 volume-%/10 volume-%/38 volume-%.

The testing was repeated at different values of the GHSV (gas hourly space velocity) of 1,550 h⁻¹ and 2100 h⁻¹.

### c) Regeneration

The regeneration was carried out by purging for 1 h with nitrogen and heating at a temperature of 550° C. in nitrogen flow. Afterwards, the flow was switched to 8 volume-% of oxygen in nitrogen. At the reactor outlet, the pressure was 5 bar(abs). The flow was continued until no CO and CO₂ were detectable. It followed a dwell time of 1 h. Thereafter the temperature of the nitrogen flow was brought to the reaction temperature and the reaction was carried out.

### Example 4.1

### Preparing P-Xylene from Methanol Using the Molding of Example 1 as the Catalyst at a GHSV of 1000 h⁻¹

The general process disclosed in Example 4 was carried out with 0.5 mL of the catalyst of Example 1 at a GHSV of 1000 h⁻¹. The p-xylene yield was measured at the third cycle of reaction (MTX3). The data are reported in Table 1.

### Comparative Example 4.1

### Preparing P-Xylene from Methanol Using the Molding of Comparative Example 1 as the Catalyst at a GHSV of 1000 h⁻¹

The general process disclosed in Example 4 was carried out with 0.5 mL of the catalyst of Comparative Example 1 at a GHSV of 1000 h⁻¹. The p-xylene yield was measured at the third cycle of reaction (MTX3). The data are reported in Table 1.

TABLE 1

| Molding | GHSV/h⁻¹ | TOS/h ^{a)} | p-xylene yield/% |
|---|---|---|---|
| E 4.1 | 1000 | 117.87 | 9.19 |
| C.E 4.1 | 1000 | 118.68 | 5.40 |

^{a)} TOS = Time on stream

### Example 4.2

### Preparing P-Xylene from Methanol at a GHSV of 1550 h⁻¹

### Example 4.2.1

### Molding of Example 2 as the Catalyst

The general process disclosed in Example 4 was carried out with 0.5 mL of the molding of Example 2 at a GHSV of 1550 h⁻¹. The p-xylene yield was measured at the third cycle of reaction. The data are reported in Table 2.

### Example 4.2.2

### Molding of Example 3 as the Catalyst

The general process disclosed in Example 4 was carried out with 0.5 mL of the molding of Example 3 at a GHSV of 1550 h⁻¹. The p-xylene yield was measured at the third cycle of reaction. The data are reported in Table 2.

### Comparative Example 4.2

### Preparing P-Xylene from Methanol Using the Molding of Comparative Example 1 as the Catalyst at a GHSV of 1550 h⁻¹

The general process disclosed in Example 4 was carried out with 0.5 mL of the molding of Comparative Example 1 at a GHSV of 1550 h⁻¹. The p-xylene yield was measured at the third cycle of reaction (MTX3). The data are reported in Table 2.

TABLE 2

| Catalyst | GHSV/h⁻¹ | TOS^{a)}/h | p-xylene yield % |
|---|---|---|---|
| E 4.2.1 | 1550 | 176.6 | 5.09 |
| E 4.2.2 | 1550 | 181.2 | 8.77 |
| C.E 4.2 | 1550 | 140.79 | 4.31 |
| C.E 4.2 | 1550 | 152.8 | 4.67 |
| C.E 4.2 | 1550 | 170.38 | 3.81 |

^{a)}TOS = Time on stream

### Example 4.3

### Preparing P-Xylene from Methanol at a GHSV of 2100 h⁻¹

### Example 4.3.1

### Molding of Example 2 as the Catalyst

The general process disclosed in Example 4 was carried out with 0.5 mL of the molding of Example 2 at a GHSV of 2100 h⁻¹. The p-xylene yield was measured at the second cycle of reaction. The data are reported in Table 3.

### Example 4.3.2

### Molding of Example 3 as Catalyst

The general process disclosed in Example 4 was carried out with 0.5 mL of the catalyst of Example 3 at a GHSV of 2100 h⁻¹. The p-xylene yield was measured at the first cycle of reaction. The data are reported in Table 3.

### Comparative Example 4.3

### Preparing P-Xylene from Methanol Using the Molding of Comparative Example 1 as catalyst at a GHSV of 2100 h⁻¹

The general process disclosed in Example 4 was carried out with 0.5 mL of the molding of Comparative Example 1 at a GHSV of 2100 h⁻¹. The p-xylene yield was measured at the first cycle of reaction (MTX3). The data are reported in Table 3.

TABLE 3

| Catalyst | GHSV/h⁻¹ | p-xylene yield/% |
|---|---|---|
| E 4.3.1 | 2100 | 9.00 |
| E 4.3.2 | 2100 | 6.04 |
| CE 4.3 | 2100 | 5.78 |

As may be taken from the results shown in Tables 1 to 3, it has surprisingly been found that the impregnation of extrudates containing ZBM or ZSM zeolitic material with P and a trivalent element such as Ga and Zn leads to an increase of the p-xylene yield with respect to the ZSM-5 zeolitic material which is first impregnated with Zn and then extrudated. Thus, as may be taken from Tables 1 to 3, the moldings of the invention display a relatively high yield with respect to a ZSM-5 zeolitic material impregnated with Zn and subsequently extrudated.

### CITED LITERATURE

U.S. Pat. No. 4,401,636
Journal of Catalysis, Vol. 147, Issue 2, June 1994, pages 482-493, “Synthesis and Characterization of ZSM-22 Zeolites and Their Catalytic Behaviour in 1-Butene Isomerization Reactions”
US 2014/0135556 A1

1.-15. (canceled)

16. A process for preparing p-xylene, comprising
(I) providing a molding comprising
(a) a zeolitic material,
(b) phosphorous,
(c) one or more metals M of the groups 3, 6, 10 to 14 of the periodic system of the elements,
(d) a binder material;
(II) providing a gas stream comprising methanol;
(III) contacting the gas stream provided in (II) with the molding provided in (1), obtaining a reaction mixture comprising p-xylene.

17. The process of claim 16, wherein the gas stream provided in (II) comprises methanol in an amount in the range of from 30 to 70 volume-% based on the total volume of the gas stream.

18. The process of claim 16, wherein the contacting according to (III) is effected at a temperature of the gas stream in the range of from 250 to 750° C.

19. The process of claim 16, wherein the contacting according to (III) is effected at a pressure of the gas stream in the range of from 1 to 100 bar(abs).

20. The process of claim 16, wherein the contacting according to (III) is carried out at a gas hourly space velocity in the range of from 500 to 3,000 h⁻¹.

21. The process of claim 16, wherein the zeolitic material has a framework structure comprising YO₂ and X₂O₃ wherein Y is a tetravalent element and X is a trivalent element.

22. The process of claim 21, wherein at least 95 weight-% of the framework structure of the zeolitic material consist of X, Y, O and H.

23. The process of claim 16, wherein the zeolitic material has a framework structure of framework type BEA, MEI, MW W, MEL, MOR, MTT, MTW, FER, TOL, or TON or wherein the zeolitic material comprises a ZSM-5 zeolitic material, a ZBM-10 zeolitic material, a ZSM-22 zeolitic material or a ZSM-11 zeolitic material.

24. The process of claim 16, wherein the molding comprises the phosphorous, calculated as elemental phosphorous, in an amount of at least 0.1 weight-% based on the total weight of the molding.

25. The process of claim 16, wherein the one or more metals M are one or more of Ga, Zn, Ni, Mo, La and Pt.

26. The process of claim 16, wherein the molding comprises the one or more metals M, calculated as elemental M, in an amount of at least 1 weight-% based on the total weight of the molding, wherein said amount refers to the total amount of all metals M.

27. The process of claim 16, wherein the one or more of metals M are comprised in the zeolitic material as extra-framework elements.

28. The process of claim 16, wherein the binder material comprises one or more of graphite, silica, titanic, zirconia, alumina, and a mixed oxide of two or more of silicon, titanium and zirconium.

29. The process of claim 16 wherein step (I) further comprises a process for preparing a molding, wherein the process comprises
(i) providing the zeolitic material
(ii) mixing the zeolitic material provided in (i) with a source of the hinder material
(iii) subjecting the mixture obtained from (ii) to molding
(iv) impregnating the molding obtained from (iii) with a source of the one or more metals M and a source of the phosphorous.

30. The process of claim 29, wherein the impregnating of (iv) is carried out by spray impregnation.
